(19) United States
(12) Patent Application Publication
(10) Pub. No.: US 2024/0100255 A1
(43) Pub. Date: Mar. 28, 2024

(54) SYRINGE DESIGN FOR CYCLING PARTICULATE SUSPENSIONS

(71) Applicant: Boston Scientific Scimed, Inc., Maple Grove, MN (US)

(72) Inventors: Ajay Gupta, Shoreview, MN (US); Randy Raymond, Maple Grove, MN (US)

(73) Assignee: Boston Scientific Scimed, Inc., Maple Grove, MN (US)

(21) Appl. No.: 18/470,272

(22) Filed: Sep. 19, 2023

Related U.S. Application Data
(60) Provisional application No. 63/410,164, filed on Sep. 26, 2022.

(51) Int. Cl. A61M 5/31; A61M 5/315

(52) U.S. Cl. CPC A61M 5/3134; A61M 5/31513

(57) ABSTRACT

The present disclosure pertains to a syringe comprising: a syringe barrel having an open proximal end that opens into an interior having a cylindrical wall and an angled distal surface terminating at an outlet; a plunger rod; and a plunger seal, a proximal end of the plunger seal configured to be attached to a distal end of the plunger rod, and a distal end of the plunger seal configured to be inserted into the interior of the syringe barrel through the open proximal end, the plunger seal forming a seal with the cylindrical wall of the interior of the syringe barrel, and the distal end of the plunger seal terminating at an angled distal surface, wherein the angled distal surface of the syringe barrel is more acute than the angled distal surface of the plunger seal. The present disclosure also pertains to kits containing such a syringe.

### CROSS-REFERENCE TO RELATED APPLICATIONS

This application claims the benefit of U.S. Provisional Patent Application Ser. No. 63/410,164 filed on Sep. 26, 2022, the disclosure of which is incorporated herein by reference.

### FIELD

This disclosure relates generally to syringes and particularly to syringes that are specifically configured to cycle particulate suspensions.

### BACKGROUND

Systems are known in which a fluid is cycled back and forth between two syringes in system in which the two syringes are linked by a connector that fluidically couples the two syringes together. The fluid may be cycled back and forth, for example, in order to thoroughly mix the fluid.

The inventors have observed that when particulate suspensions are cycled back and forth between typical off-the-shelf syringes, the particulate suspension can migrate between the plunger seal and the syringe barrel, with particulate suspension ending up on the plunger rod or inside the barrel.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In various aspects, the present disclosure pertains to a syringe comprising: (a) a syringe barrel having an open proximal end and a distal end comprising an outlet, the open proximal end of the syringe barrel opening into an interior comprising a cylindrical wall and an angled distal surface terminating at the outlet, wherein the syringe barrel has an inner diameter; (b) a plunger rod having proximal end and a distal end; and (c) a plunger seal having a proximal end and a distal end, the proximal end of the plunger seal configured to be attached to the distal end of the plunger rod, and the distal end of the plunger seal configured to be inserted into the interior of the syringe barrel through the open proximal end, the plunger seal forming a seal with the cylindrical wall of the interior of the syringe barrel, the distal end of the plunger seal terminating at an angled distal surface, wherein the angled distal surface of the syringe barrel is more acute than the angled distal surface of the plunger seal.

In some embodiments, the distal surface of the plunger seal is in the form of a right circular cone having a first angle and the angled distal surface of the syringe barrel is in the form of a right circular cone frustum having a second angle, wherein the first angle is less than the second angle. In some of these embodiments, the difference between the first angle and the second angle may range from 2° to 8°. In some of these embodiments, the first angle may range from 1200 to 160°. In some of these embodiments, the second angle may range from 1220 to 168°.

In some embodiments, which can be used in conjunction with the above aspects and embodiments, the plunger seal comprises a proximal rib having a first outer diameter and a distal rib having a second outer diameter, wherein the first outer diameter is greater than the second outer diameter. In some of these embodiments, a ratio of the first outer diameter to the second outer diameter ranges between 1.02:1 to 1.07:1. In some of these embodiments, a ratio of the first outer diameter to the inner diameter of the syringe barrel ranges between 1.10:1 to 1.05:1. In some of these embodiments, the proximal rib comprises a proximal facing surface having a first slope and a distal facing surface having a second slope, wherein the first slope is greater than the second slope. In some of these embodiments, the plunger seal further comprises one or more additional ribs between the proximal rib and the distal rib.

In some embodiments, which can be used in conjunction with the above aspects and embodiments, the proximal end of the plunger seal is provided with a threaded bore and the distal end of the plunger rod is provided with matching threads.

In some embodiments, which can be used in conjunction with the above aspects and embodiments, a plunger flange is provided at a proximal end of the plunger rod and a barrel flange is provided at the proximal end of the syringe barrel.

In some embodiments, which can be used in conjunction with the above aspects and embodiments, the plunger seal is formed from an elastomer selected from elastomer bromobutyl rubber, chlorobutyl rubber, elastomeric fluoropolymers, ethylene propylene diene monomer (EPDM) rubber, and ethylene propylene rubber (EPR).

In some embodiments, which can be used in conjunction with the above aspects and embodiments, the plunger seal comprises a low friction surface layer, such as a fluoropolymer surface layer.

Other aspects of the present disclosure pertain to kits that comprise (a) first and second syringes in accordance with any of the above aspects and embodiments and (b) a connector that fluidically connects the outlet of the first syringe to the outlet of the second syringe.

In some embodiments, the first syringe is pre-filled with particulate suspension. In some of these embodiments, the particulate suspension comprises metallic or polymeric particles. In some of these embodiments, the particulate suspension is a liquid embolic composition.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1A is a schematic illustration of a syringe design in accordance with the present disclosure.

FIG. 1B is a schematic illustration of a syringe design that is contrasts with the syringe design of FIG. 1A.

FIG. 2A is a schematic longitudinal cross-sectional view of a syringe barrel in accordance with an embodiment of the present disclosure. FIG. 2B is an enlarged view of area B in FIG. 2A.

FIG. 3 is a schematic illustration of a plunger in accordance with an embodiment of the present disclosure.

FIG. 4A is a schematic longitudinal cross-sectional view of a plunger seal in accordance with an embodiment of the present disclosure.

FIG. 4B is a schematic longitudinal cross-sectional view of a plunger seal in accordance with another embodiment of the present disclosure.

FIG. 5A is a schematic illustration of right circular cone.

FIG. 5B is a schematic illustration of right circular cone frustum.

FIG. 6 is a schematic illustration of two syringes in a coupled arrangement, in accordance with the prior art.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endovascular and endoscopic medical procedures. However, it should be noted that reference to this particular procedure is provided only for convenience and is not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

In various aspects, the present disclosure pertains to syringe designs that exhibit improved handling of particular suspensions.

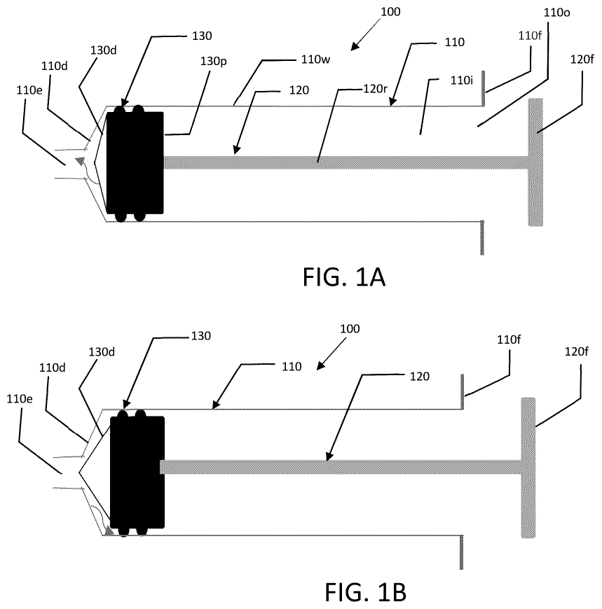

Turning now to FIG. 1A, in accordance with an embodiment of the present disclosure, a syringe 100 is shown that is made up of a syringe barrel 110, a plunger 120, and a plunger seal 130. The syringe barrel 110 has an open proximal end 110o and a distal end comprising an outlet 110e. The open proximal end 110o of the syringe barrel 110 opens into an interior 110i having a cylindrical wall 110w and an angled distal surface 110d. The plunger 120 includes a plunger rod 120r having proximal end and a distal end. At the proximal end of the plunger rod 120r is a plunger flange 120f. At the distal end of the plunger 120r is the plunger seal 130. The plunger seal 130 has a proximal end and a distal end. The proximal end 130p of the plunger seal 130 is attached to the distal end of the plunger rod 120r. The plunger seal 130 is inserted into the interior 110i of the syringe barrel 110 through the open distal end 110o of the syringe barrel. The plunger seal 130 forms a seal with the cylindrical wall 110w of the syringe barrel 110. The distal end of the plunger seal 130 terminates at an angled distal surface 130d.

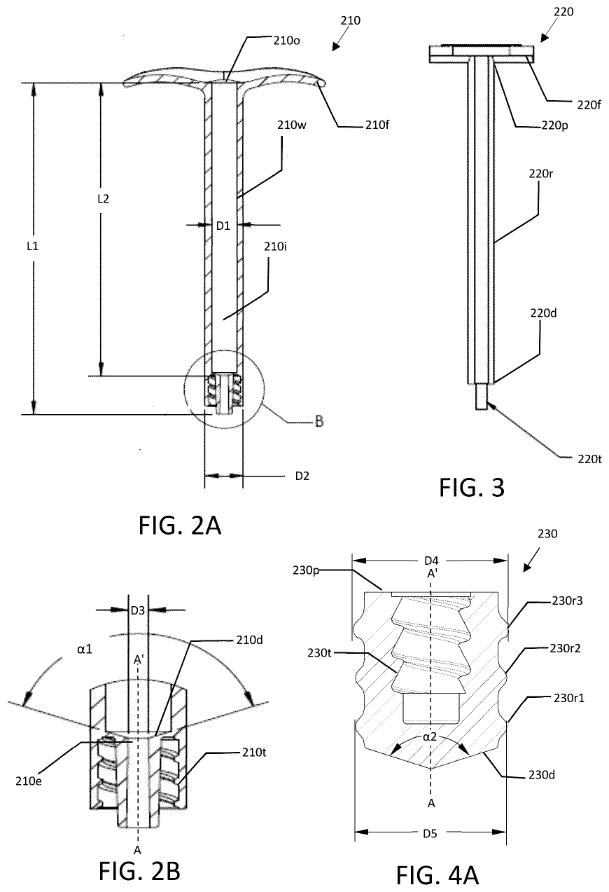

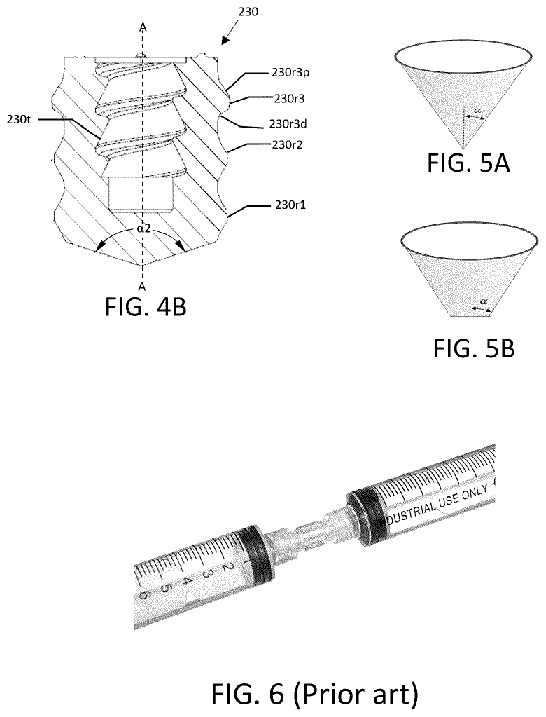

A more detailed embodiment of a syringe made up of a syringe barrel 210, a plunger 220, and a plunger seal 230 will now be described in conjunction with FIGS. 2A, 2B, 3, 4A and 4B. FIG. 2A is a schematic longitudinal cross-sectional view of a syringe barrel 210 in accordance with an embodiment of the present disclosure. FIG. 2B is an enlarged view of area B in FIG. 2A. The syringe barrel 210 shown has an open proximal end 210o and a distal end comprising an outlet 210e with a threaded exit port 210t. The open proximal end 210o of the syringe barrel 210 opens into an interior 210i having a cylindrical wall 210w and an angled distal surface 210d. As seen from FIG. 2B, an angled distal surface 210d is in the form of a partial conic surface, in particular, a surface of a frustum of a right circular cone. The distal surface 210d terminates at the outlet 210e. When viewed in a longitudinal cross-section taken along the axis A-A′ of the syringe barrel 210, the angled distal surface 210d has an angle α1. As seen by comparing FIG. 2B with FIG. 5B, the first cross-sectional angle α1 is two times the half angle α of the right circular conic frustum of the angled distal surface 210d.

The total length L1 of the syringe barrel 210 and the length L2 of the interior 210i of the syringe barrel 210 may vary widely. In certain embodiments, the total length L1 may range, for example, from 1.5 inches (3.81 cm) to 6 inches (15.2 cm) and the length L2 may range, for example, from 1.1 inches (2.79 cm) to 5.6 inches (14.2 cm). Similarly, the inner diameter D1 of the interior 210i of the syringe barrel 210 and the inner diameter D3 of the outlet 210e may vary widely. In certain embodiments, the diameter D1 may range, for example, from 0.25 inch (0.64 cm) to 1.5 inches (3.81 cm). The outer diameter D2 of the syringe barrel 210 will be somewhat larger than D1 and will vary based on material properties and ability to mold deep drawn, thin-walled parts.

FIG. 3 is a schematic illustration of an embodiment of a plunger 220 in accordance with an embodiment of the present disclosure. The plunger 220 includes a plunger rod 220r having proximal end and a distal end. At the proximal end 220p of the plunger rod 220r is a plunger flange 220f. At the distal end 220d of the plunger rod 220r is a distal portion 220t of the plunger that engages with the plunger seal 230. Although not specifically illustrated, the distal portion 220t may be threaded to engage complementary threads in a bore in the plunger seal 230 as described below.

FIG. 4A is a schematic longitudinal cross-sectional view of a plunger seal 230 in accordance with an embodiment of the present disclosure. The plunger seal 230 has a proximal end 230p and a distal end terminating at an angled distal surface 230d. The proximal end 230p of the plunger seal 230 is configured for attachment to the distal end 220d of the plunger rod 220r. In the embodiment shown, the proximal end 230p of the plunger seal 230 is provided with a threaded bore 230t. Although not specifically illustrate in FIG. 3, the distal end 220d of the plunger rod 220 may be provided with threads 220t to match those of the threaded bore 230t.

The plunger seal 230 of FIG. 4A is configured to be inserted into the interior 210i of the syringe barrel 210 shown in FIGS. 2A and 2B through the open proximal end 210o of the syringe barrel 210. The plunger seal 230 forms a seal with the cylindrical wall 210w of the syringe barrel 210. The distal end of the plunger seal 230 terminates at an angled distal surface 230d. As seen from FIG. 4A, the angled distal surface 230d is in the form of a conic surface, in particular, a surface of a right circular cone. When viewed in a longitudinal cross-section taken along the axis A-A′ of the plunger seal 230, the angled distal surface 230d has an angle α2. As seen by comparing FIG. 4A with FIG. 5A, the first cross-sectional angle α2 is two times the half angle α of the circular conic shape of the angled distal surface 230d.

The plunger seal 230 in the embodiment of FIG. 4A further contains three plunger seal ribs: a distal plunger seal rib 230rl, a middle plunger seal rib 230r2, and a proximal plunger seal rib230r3. While one central plunger seal rib 230r2 is shown, zero, two, three, or more central plunger seals may be present.

The syringes shown in the preceding figures are particularly well adapted for use in ejecting particulate suspensions from the syringe barrel. Myriad particulate suspensions are known in a wide variety of fields which may be used in conjunction with the syringes of the present disclosure. However, a specific application will now be described which pertains to injectable compositions that contain a suspension of, for example, metallic radiopaque particles (e.g., tantalum, palladium or gold) or polymeric radiopaque particles (e.g., polymer particles containing covalently bound iodine atoms). One particular example is a liquid embolic composition in the form of a particulate suspension that contain tantalum particles in a pre-filled syringe. The particles of such particulate suspensions tend to settle when quiescent. One way to homogenize a particulate suspension is to connect an empty syringe to the pre-filled syringe using a connector having a lumen extending therethrough, which connect the outlets of the two syringes together in an arrangement like that shown in FIG. 6. Once connected, the particulate suspension can be cycled or “ping-ponged” between the two syringes. This arrangement can dramatically improve ease of homogenization.

The inventors have found that typical off-the-shelf plungers and syringe barrels are effective at containing particular suspensions and preventing leaks during autoclaving and storage. However, as the particulate suspension is cycled between the syringes, the particulate suspension has been observed to migrate proximally between the plunger seal and the syringe barrel, ending up on the plunger rod or inside the barrel. The present disclosure provides multiple innovations to address this deficiency.

One innovation that has been found to decrease the migration of a particulate suspension past the plunger seal 230 is to ensure that the angle α1 of the angled distal surface 210d of the syringe barrel 210 is less than the angle α2 of the distal surface 230d of the plunger seal 230. When the plunger seal 230 is bottomed out in the syringe barrel 210, this will force the particulate suspension to the syringe tip, instead of simply compressing it or forcing it towards the plunger seal 230.

This can be seen with reference again to FIG. 1A and to FIG. 1B. In FIG. 1A, the angled distal surface 110d of the syringe barrel 110 has an angle that is less than an angle of the angled distal surface 130d of the plunger seal 130. As shown by the arrow in FIG. 1A, this difference in angles acts to urge all of the contents that are present in the syringe barrel 110 distally toward the outlet 110e of the syringe barrel 110.

FIG. 1B is identical to FIG. 1A, except that the angled distal surface 110d of the syringe barrel 110 has an angle that is greater than an angle of the angled distal surface 130d of the plunger seal. While a majority of the contents that are present in the syringe barrel 110 are pushed distally toward the outlet 110e of the syringe barrel 110, as shown by the arrow in FIG. 1B, a portion of the fluid (or fluid suspension) that is present in the syringe 100 is urged proximally toward the open proximal end 110o of the syringe barrel 110.

With reference again to FIG. 2A, FIG. 2B, FIG. 3, FIG. 4A and FIG. 4B, and specifically with reference to FIG. 2A, FIG. 2B and FIG. 4A, the angle α1 of the angled distal surface 210d of the syringe barrel 210 typically ranges from 1200 to 160°, beneficially ranging from 1320 to 152°. The angle α2 of the distal surface 230d of the plunger seal 230 typically ranges from 1220 to 168°, beneficially ranging from 1360 to 156°. The difference between the angle α1 and the angle α2 typically ranges from about 2° to 8°, beneficially ranging from 3° to 5°. It should be noted that angles α1 and α2 can vary to a larger degree based on ease of molding and aesthetics, among other reasons.

Another innovation that was found to decrease the movement of a particulate suspension past the plunger seal is that the outer diameter of the proximal-most rib (e.g., diameter D4 of the proximal plunger seal rib 230r3 in FIG. 4A) is increased relative to a diameter of the remaining ribs (e.g., diameter D5 of both the distal plunger seal rib 230r1 and the central plunger seal rib 230r2). This acts to increase compression of the proximal plunger seal rib 230r3 within the syringe barrel 210, giving a tighter seal. In some embodiments, a ratio of the diameter D4 to D5 may range, for example, from 1.02:1 to 1.07:1.

Relative to the inner diameter D1 of the syringe barrel 210, a ratio of the diameter D4 to the inner diameter D1 may range, for example, from 1.10:1 to 1.05:1, and a ratio of the diameter D5 to the inner diameter D1 may range, for example, from 1.08:1 to 1.03:1. These ratios are also known as compression ratios. The higher the compression ratio, the more the friction (making it difficult to move the plunger seal) and the tendency to deform the ribs, which may cause particles to leak beyond the ribs. In various embodiments, the ribs have an outer surface curvature comprising a radius ranging from 0.012 inch (0.030 cm) to 0.025 inch (0.063 cm), more typically ranging from 0.015 inch (0.038 cm) to 0.02 inch (0.51 cm).

In a particular embodiment, the diameter D4 of the proximal plunger seal rib230r3 may be 0.274 inch (0.696 cm), the diameter D5 of the distal plunger seal rib 230r1 and middle plunger seal rib 230r2 may be about 0.266 inch (0.676 cm), and the diameter D1 of the syringe barrel 210 may be about 0.253 inch (0.643 cm).

Still another innovation is the use of a so-called “swept forward design” for the proximal plunger seal rib 230r3, which helps to push any material that escapes the distal plunger seal rib 230r1 (and any central plunger seal rib 230r2) towards the front of the syringe. This design can be seen in FIG. 4B, wherein the plunger seal rib 230r3 is asymmetric in that the distal face 230r3d of the proximal plunger seal rib 230r3 is provided with first slope and the proximal face 230r3p of the proximal plunger seal rib 230r3 is provided with a second slope that is greater than the first slope. For example, the distal face 230r3d of the proximal plunger seal rib 230r3 may have increased concavity and/or increased undercut relative to the proximal face 230r3p of the proximal plunger seal rib 230r3. In some embodiments, the proximal face 230r3p of the proximal plunger seal rib 230r3 may have a linear taper. By providing less slope or undercut for the proximal face 230r3p of the proximal plunger seal rib 230r3, the tendency of the proximal plunger seal rib 230r3 to buckle may be minimized.

A variety of materials may be used to construct the plunger seals described herein. For example, the plunger seals may be formed using a suitable elastomer such as bromobutyl rubber, chlorobutyl rubber, elastomeric fluoropolymers, ethylene propylene diene monomer (EPDM) rubber, and ethylene propylene rubber (EPR), among other possibilities. The plunger seals may be formed using methods known in the art. As one example, in a first step an elastomer may be formulated in a roll mill and the resulting sheet calendared. A piece of the sheet may then be placed in a mold (e.g., a metal mold), and the plunger seal made by compression molding, during which the elastomer is forced into the cavities of the mold and, where reactive, undergoes a full curing reaction. After the plunger sea is molded, the parts can be post-processed, for example, by removing flash and gate vestige and can optionally be cleaned and undergo surface treatment, for example, by optionally coating or surface treatment of the plunger seal with a fluoropolymer to reduce friction forces.

A variety of materials may be used to construct the syringe barrels described herein. Typical materials for using in constructing the syringe barrels described herein include plastics such as polycarbonate, polypropylene, polyethylene, nylon copolymers, copolyesters, urethanes, cyclic olefins, polymethylpentene; glass; metals such as stainless steel, and other materials known in the art. A variety of materials may be used to construct the plungers described herein. Typical materials for using in constructing the plungers described herein include rigid plastics (either unfilled or reinforced with glass fibers or fillers, glass, metals and other materials known in the art).

Other aspects of the present disclosure pertain to kits that contain syringes in accordance with the present disclosure. For example, the kits may comprise an empty syringe in accordance with the present disclosure and a syringe in accordance with the present disclosure that is prefilled with a particulate suspension. Examples of particulate suspensions vary widely as previously discussed. In one particular embodiment, the particulate suspension may be a suspension of metallic or polymeric radiopaque particles in an injectable composition, for example, a liquid embolic composition. The kits may further comprise a connector for fluidically coupling two syringes together. For example, the connector may comprise a first port that is configured to provide a fluid connection with the exit port of the empty syringe, a second port that is configured to provide a fluid connection with the exit port of the prefilled syringe, and a lumen fluidically connecting the first and second ports. For example, the first and second ports may include a coupling mechanism such as a plurality of threads for coupling to the threaded exit port of the syringe barrel described herein.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Any reference signs in the claims are provided merely as a clarifying example and shall not be construed as limiting the scope of the claims in any way.

1. A syringe comprising:
a syringe barrel having an open proximal end and a distal end comprising an outlet, the open proximal end of the syringe barrel opening into an interior comprising a cylindrical wall and an angled distal surface terminating at the outlet, wherein the syringe barrel has an inner diameter;
a plunger rod having proximal end and a distal end; and
a plunger seal having a proximal end and a distal end, the proximal end of the plunger seal configured to be attached to the distal end of the plunger rod, and the distal end of the plunger seal configured to be inserted into the interior of the syringe barrel through the open proximal end, the plunger seal forming a seal with the cylindrical wall of the interior of the syringe barrel, the distal end of the plunger seal terminating at an angled distal surface;
wherein the angled distal surface of the syringe barrel is more acute than the angled distal surface of the plunger seal.

2. The syringe of claim 1, wherein the distal surface of the plunger seal is in the form of a right circular cone having a first angle and wherein the angled distal surface of the syringe barrel is in the form of a right circular cone frustum having a second angle, wherein the first angle is less than the second angle.

3. The syringe of claim 2, wherein the difference between the first angle and the second angle ranges from 2° to 8°.

4. The syringe of claim 2, wherein the first angle ranges from 1200 to 160°.

5. The syringe of claim 3, wherein the second angle ranges from 122° to 168°.

6. The syringe of claim 1, wherein the plunger seal comprises a proximal rib having a first outer diameter and a distal rib having a second outer diameter, wherein the first outer diameter is greater than the second outer diameter.

7. The syringe of claim 6, wherein a ratio of the first outer diameter to the second outer diameter ranges between 1.02:1 to 1.07:1.

8. The syringe of claim 6, wherein a ratio of the first outer diameter to the inner diameter of the syringe barrel ranges between 1.10:1 to 1.05:1.

9. The syringe of claim 6, wherein the proximal rib comprises a proximal facing surface having a first slope and a distal facing surface having a second slope, wherein the first slope is greater than the second slope.

10. The syringe of claim 6, wherein the plunger seal further comprises one or more additional ribs between the proximal rib and the distal rib.

11. The syringe of claim 1, wherein the proximal end of the plunger seal is provided with a threaded bore and the distal end of the plunger rod is provided with matching threads.

12. The syringe of claim 1, wherein a plunger flange is provided at a proximal end of the plunger rod and a barrel flange is provided at the proximal end of the syringe barrel.

13. The syringe of claim 1, wherein the plunger seal is formed from an elastomer selected from elastomer bromobutyl rubber, chlorobutyl rubber, elastomeric fluoropolymers, ethylene propylene diene monomer (EPDM) rubber, and ethylene propylene rubber (EPR).

14. The syringe of claim 1, wherein the syringe barrel is formed from a polymer selected from polycarbonate, polypropylene, polyethylene, nylon copolymers, copolyesters, polyurethanes, cyclic olefin polymers, and polymethylpentene.

15. The syringe of claim 1, wherein the syringe barrel is formed from a rigid plastic that is optionally reinforced with a particulate filler.

16. The syringe of claim 1, wherein the plunger seal comprises a fluoropolymer surface layer.

17. A syringe comprising:
a syringe barrel having an open proximal end and a distal end comprising an outlet, the open proximal end of the syringe barrel opening into an interior comprising a cylindrical wall and an angled distal surface terminating at the outlet, wherein the syringe barrel has an inner diameter;
a plunger rod having proximal end and a distal end; and
a plunger seal comprising a proximal end, a distal end, a proximal rib having a first outer diameter and a distal rib having a second outer diameter, the first outer diameter being greater than the second outer diameter, the proximal end of the plunger seal configured to be attached to the distal end of the plunger rod, the distal end of the plunger seal configured to be inserted into the interior of the syringe barrel through the open proximal end, the plunger seal forming a seal with the cylindrical wall of the interior of the syringe barrel, the distal end of the plunger seal terminating at an angled distal surface;
wherein the angled distal surface of the syringe barrel is more acute than the angled distal surface of the plunger seal, wherein the distal surface of the plunger seal is in the form of a right circular cone having a first angle, and wherein the angled distal surface of the syringe barrel is in the form of a right circular cone frustum having a second angle, wherein the first angle is less than the second angle.

18. A kit comprising (a) first and second syringes, each of the first and second syringes comprising: a syringe barrel having an open proximal end and a distal end comprising an outlet, the open proximal end of the syringe barrel opening into an interior comprising a cylindrical wall and an angled distal surface terminating at the outlet, wherein the syringe barrel has an inner diameter; a plunger rod having proximal end and a distal end; a plunger seal having a proximal end and a distal end, the proximal end of the plunger seal configured to be attached to the distal end of the plunger rod, and the distal end of the plunger seal configured to be inserted into the interior of the syringe barrel through the open proximal end, the plunger seal forming a seal with the cylindrical wall of the interior of the syringe barrel, the distal end of the plunger seal terminating at an angled distal surface; wherein the angled distal surface of the syringe barrel is more acute than the angled distal surface of the plunger seal; and (b) a connector that fluidically connects the outlet of the first syringe to the outlet of the second syringe.

19. The kit of claim 18, wherein the first syringe is pre-filled with particulate suspension.

20. The kit of claim 19, wherein the particulate suspension is a liquid embolic composition.
